(19) United States
(12) Patent Application Publication
(10) Pub. No.: US 2008/0082046 A1
(43) Pub. Date: Apr. 3, 2008

(54) Balloon catheter, an assembly of the balloon catheter and a method of inserting the assembly into a guiding catheter

(75) Inventors: Tomihisa Kato, Aichi (JP); Fuminobu Yoshimachi, Aomori (JP)

Correspondence Address: MORGAN LEWIS & BOCKIUS LLP, 1111 PENNSYLVANIA AVENUE NW, WASHINGTON, DC, 20004, US

(73) Assignees: Asahi Intecc Co., Ltd.; Filmecc Co., Ltd.

(21) Appl. No.: 11/898,769

(22) Filed: Sep. 14, 2007

(30) Foreign Application Priority Data
Sep. 29, 2006 (JP) 2006-269225

(51) Int. Cl. A61M 25/10

(52) U.S. Cl. 604/101.01; 604/103.04; 604/96.01

(57) ABSTRACT

In an assemble of a balloon catheters (2, 3), there are provided an elongate lumen tube (21, 31), a balloon portion (22, 32) provided at a distal end of the elongate lumen tube (21, 31) and a terminal tool (23, 33) placed at proximal ends of the balloon catheters (2, 3). The balloon catheters (2, 3) have inner tubes (25, 35) into which a guide wire (10) is inserted, and having outer tubes (27, 37) around the inner tubes (25, 35) to be in communication with balloons (B) of the balloon portions (22, 32). The elongate lumen tubes (21, 31) join the balloon catheters (2, 3) together in integral therewith. Latitudinal cross sections of the outer tubes (27, 37) are substantially a semi-circular, fan-like or trapezoidal or oval configuration, otherwise the cross sections are a trapezoidal or rectangular configuration with one lateral side deformed into an arcuate configuration. This enables users to make an effective use of an interior lumen of a guiding catheter (4), thus dimensionally downsizing the balloon catheters (2, 3) while keeping an outer diameter of the balloon (B) and a thickness of the guiding catheter (4) the same size as larger ones.

### BACKGROUND OF THE INVENTION

1. Field of the Invention

The invention relates to an assemble of balloon catheters and the balloon catheter used to combining the balloon catheters which are suited to therapeutically dilating an occlusive area at a branched portion of the blood vessel by means of a kissing operation.

2. Description of Prior Art

Upon therapeutically dilating an occlusive area at a branched blood vessel, a guiding catheter is firstly inserted in the blood vessel, and a plurality of balloon catheters are placed within the guiding catheter to be led to a target area in the blood vessel. The kissing operation enables a user to concurrently inflate the two balloons of the balloon catheters inserted into the occlusive area in advance. It has been a tendency of the guiding catheter to be dimensionally downsized from 8F size to 6F size for implementing the kissing operation.

A thinned guiding catheter enables the user to quicken an arrest of hemorrhage with a limited incision, and reducing a usable amount of the contrast medium without having a harmful affect on the renal function, thus mitigating a burden of patients, and responding to the demand for a less intrusive (unobtrusive) operation.

For this reason, it is desirable to use the guiding catheter, an outer diameter of which is 1.66 mm (5F) or 1.33 mm (4F) which are far less than 2.04 mm (6F) for implementing the kissing operation.

When the reduced outer diameter of the guiding catheter, however, accompanies a decline of thickness of the guiding catheter to gain the rigidity of the balloon catheter, it poses a problem to make the kissing operation difficult.

As an example of the balloon catheter used for implementing the kissing operation, Japanese Laid-open Patent Application No. 2005-28118 discloses a balloon portion processed with a lubricious treatment, and improving a gripping and crossing property. Japanese Laid-open Patent Application No. 2-13470 discloses a stepped balloon structure provided as a damage-preventing instrument for a bifurcated blood vessel. Japanese Published Patent Application No. 2003-525065 discloses a bifurcation stent delivery system in which a stepped balloon is used to deploy a stent at a diseased portion of the bifurcated blood vessel. None of those references discloses an assemble of balloon catheters which contributes to dimensionally downsize the balloon catheters with an increased rigidity maintained.

It is an object of the invention to respond to the recent demand of the user, and provide an assemble of balloon catheters and the balloon catheter which are capable of maximumly making an effective use of an interior lumen of a guiding catheter by contriving an exotic balloon catheter, thus making it possible to dimensionally downsize the balloon catheter with an increased rigidity while keeping an outer diameter of the balloon portion and a thickness of the guiding catheter the same size as larger ones.

### SUMMARY OF THE INVENTION

According to the invention, there is provided a balloon catheter having an elongate lumen tube, a balloon portion provided at a distal end of the elongate lumen tube and a terminal tool provided at a proximal end of the balloon catheter, so as to be inserted into a guiding catheter which is to be placed in a blood vessel when in use. The balloon catheters form over-the-wire balloon catheters in which the elongate lumen tube has an inner tube into which a guide wire is inserted, and having an outer tube provided around the inner tube to be in communication with a balloon of the balloon portion. A latitudinal cross sectional shape of the outer tube is such as to partly form an arcuate side generally having a curvature near to a curvature of an inner wall of the guiding catheter, and partly forming a linear side at a central portion opposite to the arcuate side.

With the latitudinal cross section of the outer tube formed into a semi-circular, fan-like, oval or elliptical configuration, otherwise formed into trapezoid or rectangular configuration with one lateral side deformed arcuately, while the cross section partly forming an arcuate side generally having a curvature near to a curvature of an inner wall of the guiding catheter, it is possible to maximumly make an effective use of an interior lumen of a guiding catheter, thus dimensionally downsizing the balloon catheter positively with an increased rigidity while keeping an outer diameter of the balloon portion and a thickness of the guiding catheter the same size as larger ones.

It is to be noted that the arcuate side may have a curvature approximate to that of the inner wall of the guiding catheter, instead of having an exact curvature to that of the inner wall of the guiding catheter, and the linear side may have undulating portions, instead of having an exact linearity.

According to other aspect of the invention, there is provided a balloon catheter having an elongate lumen tube, a balloon portion provided at a distal end of the elongate lumen tube and a terminal tool provided at a proximal end of the balloon catheter, so as to be inserted into a guiding catheter which is to be disposed in the blood vessel when in use. The balloon catheters form a rapid-exchange balloon catheters in which a distal end side of the elongate lumen tube has an inner insertion opening for a guide wire, an inner space of the elongate lumen tube forms an interior lumen to admit the guide wire therein. The elongate lumen tube has an inner tube, a distal end of which serves as an exit opening for the guide wire, and having an outer tube coaxially arranged around the inner tube. A distal end side of the outer tube has an outer insertion opening for the guide wire which is to be in communication with the inner insertion opening for the guide wire and a balloon of the balloon portion. A latitudinal cross section of the outer tube is such as to partly form an arcuate side generally having a curvature near to a curvature of an inner wall of the guiding catheter, and partly forming a linear side at a central portion opposite to the arcuate side.

With the latitudinal cross section of the outer tube formed into a semi-circular, fan-like, oval or elliptical configuration, otherwise formed into trapezoidal or rectangular configuration with one lateral side deformed arcuately, while the cross section partly forming an arcuate side generally having a curvature near to a curvature of an inner wall of the guiding catheter, it is possible to maximumly make an effective use of an interior lumen of a guiding catheter, thus dimensionally downsizing the balloon catheter positively with an increased rigidity while keeping an outer diameter of the balloon portion and a thickness of the guiding catheter the same size as larger ones.

It is to be appreciated that the arcuate side may have a curvature approximate to that of the inner wall of the guiding catheter, instead of having an exact curvature to that of the inner wall of the guiding catheter, and the linear side may have undulating portions, instead of having an exact linearity.

According to other aspect of the invention, there is provided an assemble of the balloon catheter having two balloons in which the two elongate lumen tubes are slidably joined to form a conjugate lumen tube. The outer tubes of the two elongate lumen tubes are generally symmetrical or identical (e.g., elliptical or oval) each other, and latitudinal cross sections of the two elongate lumen tubes are such that the outer tubes make the arcuate side slidable against or located near the inner wall of the guiding catheter. The conjugate lumen tube makes one linear side of the outer tubes abut against the other linear side of the outer tubes. The conjugate lumen tube has an elongate portion (including balloon portion) jutted out of the guiding catheter, and making the two outer tubes slidably joined in the axial direction within the guiding catheter while making the elongate portion separable each other.

The above structure enables the user to maximize an effective use of the interior lumen of the guiding catheter, thus rendering it possible to apply the balloon catheter in which the outer tubes has a larger cross sectional area.

The two outer tubes are slidably joined at their linear sides which serves as reinforcement ribs to strengthen the rigidity of the assemble of the balloon catheter. This makes the kissing operation easy, and dimensionally downsizes the balloon catheter positively while keeping an outer diameter of the balloon portion and a thickness of the guiding catheter the same size as larger ones.

According to other aspect of the invention, the conjugate lumen tube located at least within the guiding catheter is formed by linearly juxtapositionally joining or helically joining the two elongate lumen tubes.

The above structure forms a prop wall at a joined portion between the elongate lumen tubes, and gains the rigidity of the assemble of the balloon catheters to strengthen a moment of inertia, thus making the kissing operation easy with a good pushability and following-up capability.

According to other aspect of the invention, helically joining number of the two elongate lumen tubes is 1-3 times.

With the two elongate lumen tubes twisted 1-3 times in a spiral fashion, it is possible to incorporate the two elongate lumen tubes into one united body, thus making it easy to insert the assemble of the balloon catheters into the guiding catheter.

Upon moving one of the balloon catheters forward or rearward relatively with the rest of the balloon catheters retained stationary, it is possible to protract or retract a distal end portion of the one balloon catheter against the guiding catheter. This makes it possible to advantageously advance or retract the distal end portion of the one balloon catheter into or from a diseased area in the blood vessel while rotationally moving the distal end portion of the one balloon catheter.

According to other aspect of the invention, both the balloon catheters are the over-the-wire balloon catheters. Latitudinal cross sections of the outer tubes is formed into a semi-circular, oval or elliptical configuration.

The above combination of the balloon catheters enables the user to make an effective use of the latitudinal cross section of the guiding catheter so as to insure the same useful advantages as described hereinbefore. It is especially practical for the outer tubes to have the semi-circular, oval or elliptical cross section.

According to other aspect of the invention, one of the two balloon catheters is the over-the-wire balloon catheter with the latitudinal cross section of the outer tube formed into a fan-shaped configuration, and the rest of the two balloon catheters is the rapid-exchange balloon catheter with the latitudinal cross section of the outer tube formed into a fan-shaped configuration.

In addition to the same useful advantages as described hereinbefore, the structure enables the user to implement the kissing operation through the properties characteristic of the over-the-wire balloon catheter and the rapid-exchange balloon catheter.

According to other aspect of the invention, the two balloon catheters are rapid-exchange balloon catheters, and latitudinal cross sections of the outer tubes are generally formed into a trapezoidal configuration with one lateral side shaped arcuately.

In addition to the same useful advantages as described hereinbefore, the structure enables the user to attain a delivery path having a larger cross sectional area for the contrast medium.

According to other aspect of the invention, the balloon portions of the two balloon catheters are juxtaposed in a front-and-rear relationship with each other, so as to prevent the balloon portions from being overlapped side by side at a common position.

Since the balloon portions are diametrically greater than the elongate lumen tubes, when the balloon portions overlap side by side at the common position, the overlapped portion gains a greater diameter to make it difficult to insert the balloon portions into the guiding catheter of smaller diameter.

With the balloon portions prevented from being overlapped side by side, it is possible to diametrically reduce the balloon portions so as to readily enable the user to insert the balloon portions into the guiding catheter of smaller diameter.

According to other aspect of the invention, a linearly juxtapositionally joining portion or a helically joining portion of the elongate lumen tubes is formed by the same synthetic resin of polytetrafluoroethylene.

Polytetrafluoroethylene has a exceedingly small static coefficient of friction among the same kind of sliding synthetic members. By forming the outer tubes of the balloon catheters with polytetrafluoroethylene, it is possible to reduce the static coefficient of friction between the outer tubes, so as to enhance a mutual sliding property therebetween to smoothly implement the kissing operation. This holds true when producing the outer tubes by coating polytetrafluoroethylene on a polyamide tube. The identical kind of synthetic applied for the outer tubes reduces a manufacturing cost with a simplified maintenance, compared to the case in which two balloon catheters have the outer tubes of different material.

According to other aspect of the invention, an outer diameter of the guide wire is 0.254-0.355 mm (0.010-0.014 inches); and an outer diameter of the guiding catheter is approximately 1.66 mm {0.065 inches (5F)}. This makes it possible to produce the assemble of the 5F-sized balloon catheters which enables the user to adopt the 5F-sized guiding catheter.

According to other aspect of the invention, an outer diameter of the guide wire is 0.203-0.254 mm (0.008-0.010 inches), and an outer diameter of the guiding catheter is approximately 1.33 mm {0.052 inches (4F)}. This makes it possible to produce the assemble of the 5F-sized balloon catheters which enables the user to adopt the 4F-sized guiding catheter.

According to other aspect of the invention, there is provided a method of inserting an assemble of balloon catheters into a guiding catheter. A guiding catheter is inserted into a blood vessel along two guide wires inserted into the blood vessel with proximal end sides of the two guide wires extracorporeally exposed. The two balloon catheters inserted to outer surfaces of the two guide wires from the corresponding distal ends of the balloon portions through the two guide wires exposed at a proximal end side of the guiding catheter. The two elongate lumen tubes of the balloon portions are linearly juxtapositionally joined or helically joined partly or wholly to form the conjugate lumen tube. The conjugate lumen tube is inserted into the guiding catheter after forming the conjugate lumen tube.

The method enables the user to form the conjugate lumen tube concurrently at the time of inserting the assemble of balloon catheters into the guiding catheter, so as to reduce the time period needed to implement the therapeutical operation.

Upon forming the conjugate lumen tube, it is shaped from a distal end side of the lumen tubes of the two balloon catheters by gradually inserting the balloon catheters into the guiding catheter while progressively shaping the conjugate lumen tube.

Otherwise, the two balloon catheters may be juxtapositionally joined or helically joined to form the conjugate lumen tube beforehand, and inserted into the guiding catheter through a distal end side thereof.

### BRIEF DESCRIPTION OF THE DRAWINGS

Preferred forms of the present invention are illustrated in the accompanying drawings in which:

FIG. 1 is an enlarged side elevational view of an assemble of balloon catheters surrounded by a circle F of FIG. 2 according to a first embodiment of the invention but partly broken;

FIGS. 2 and 3 are side elevational views of the assemble of balloon catheters which is to be inserted into a guiding catheter;

FIG. 4 is an enlarged side elevational view surrounded by a circle E of FIG. 2;

FIG. 5 is an enlarged side elevational view surrounded by a circle G of FIG. 3;

FIG. 6 is an enlarged latitudinal cross sectional view looked from a severed open end of the conjugate lumen tube in FIG. 5;

FIG. 7 is an enlarged side elevational view of an assemble of balloon catheters surrounded by a circle H of FIG. 8;

FIGS. 8 and 9 are side elevational views of the assemble of balloon catheters inserted into a guiding catheter;

FIG. 10 is an enlarged side elevational view of an assemble of balloon catheters surrounded by a circle I of FIG. 8;

FIG. 11 is an enlarged side elevational view of an assemble of balloon catheters surrounded by a circle J of FIG. 9;

FIG. 12 is an enlarged latitudinal cross sectional view of a guide wire, an inner tube and an outer tube;

FIG. 13 is an enlarged latitudinal cross sectional view of a guide wire, an inner tube and an outer tube according to a second embodiment of the invention;

FIG. 14 is an enlarged latitudinal cross sectional view of a guide wire, an inner tube and an outer tube according to a third embodiment of the invention;

FIG. 15 is an enlarged latitudinal cross sectional view of a prior guide wire, inner tube and outer tube;

FIG. 16 is an enlarged latitudinal cross sectional view of a guide wire, an inner tube and an outer tube according to a fourth embodiment of the invention;

FIG. 17 is an enlarged latitudinal cross sectional view of a guide wire, an inner tube and an outer tube according to a fifth embodiment of the invention;

FIGS. 18 through 20 are schematic views showing how a balloon portion works at a bifurcated blood vessel;

FIG. 21 is an enlarged side elevational view of an assemble of balloon catheters surrounded by a circle K of FIG. 22 according to the fourth embodiment of the invention but partly broken;

FIGS. 22 and 23 are side elevational views of the assemble of balloon catheters;

FIG. 24 is an enlarged side elevational view surrounded by a circle L of FIG. 22;

FIG. 25 is an enlarged side elevational view surrounded by a circle M of FIG. 23;

FIG. 26 is an enlarged latitudinal cross sectional view looked from a severed open end of the conjugate lumen tube in FIG. 25;

FIG. 27 is an enlarged latitudinal cross sectional view taken along lines P1-P1 of FIG. 28;

FIG. 28 is an enlarged longitudinal cross sectional view of the outer tube;

FIG. 29 is an enlarged latitudinal cross sectional view taken along lines P2-P2 of FIG. 28;

FIG. 30 is an enlarged side elevational view of an assemble of balloon catheters surrounded by a circle Q of FIG. 31 according to the fourth embodiment of the invention but partly broken;

FIGS. 31 and 32 are side elevational views of the assemble of balloon catheters inserted into a guiding catheter;

FIG. 33 is an enlarged side elevational view surrounded by a circle R of FIG. 31;

FIG. 34 is an enlarged side elevational view surrounded by a circle S of FIG. 32;

FIG. 35 is an enlarged side elevational view of an assemble of balloon catheters surrounded by a circle T of FIG. 36 according to the fifth embodiment of the invention but partly broken;

FIGS. 36 and 37 are side elevational views of the assemble of balloon catheters which is to be inserted into a guiding catheter;

FIG. 38 is an enlarged side elevational view surrounded by a circle U of FIG. 36;

FIG. 39 is an enlarged side elevational view surrounded by a circle V of FIG. 37;

FIG. 40 is an enlarged latitudinal cross sectional view looked from a severed open end of the conjugate lumen tube in FIG. 39;

FIG. 41 is an enlarged side elevational view of an assemble of balloon catheters surrounded by a circle W of FIG. 42 according to the fifth embodiment of the invention but partly broken;

FIGS. 42 and 43 are side elevational views of the assemble of balloon catheters inserted into a guiding catheter;

FIG. 44 is an enlarged side elevational view surrounded by a circle X of FIG. 42;

FIG. 45 is an enlarged side elevational view surrounded by a circle Y of FIG. 43;

FIGS. 46 and 47 are schematic views showing how the assemble of balloon catheters navigates its distal end portion in the blood vessel; and

FIG. 48 is a schematic view showing how the assemble of balloon catheters is manipulatively inserted into the blood vessel of a human body.

### DETAILED DESCRIPTION OF THE PREFERRED EMBODIMENTS

In the following description of the depicted embodiments, the same reference numerals are used for features of the same type.

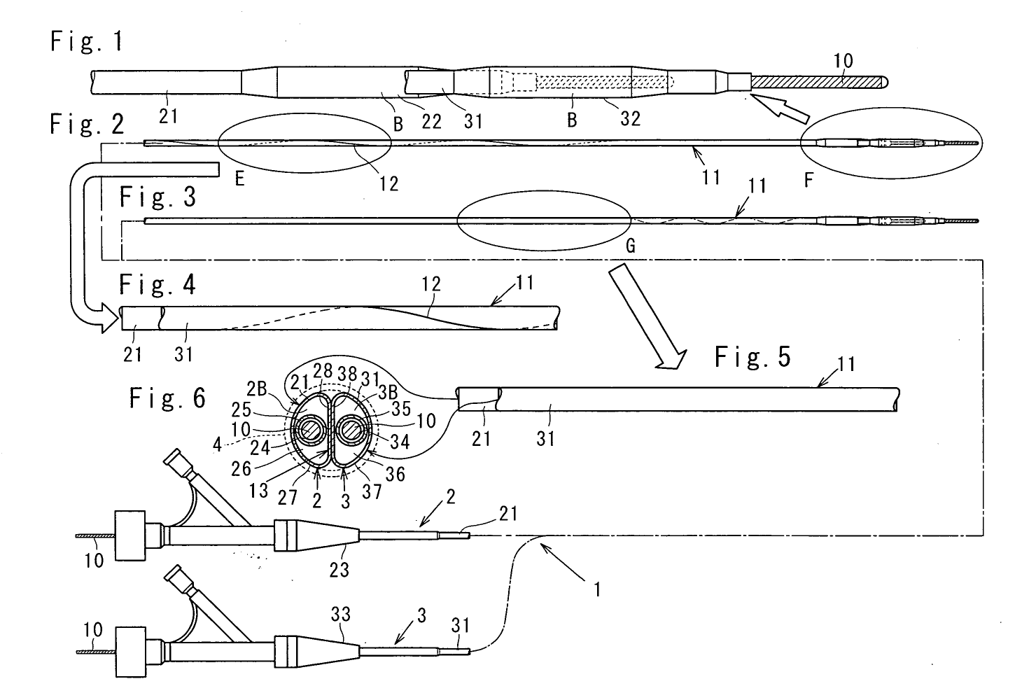

Referring to FIGS. 1 through 6 which show an assemble 1 of catheters according to a first embodiment of the invention, two balloon catheters 2, 3, which has a generally identical structure, are combined to form a united single structure. Each of the two balloon catheters 2, 3 is an over-the-wire balloon catheter, a basic structure of which is known well-versed in the art.

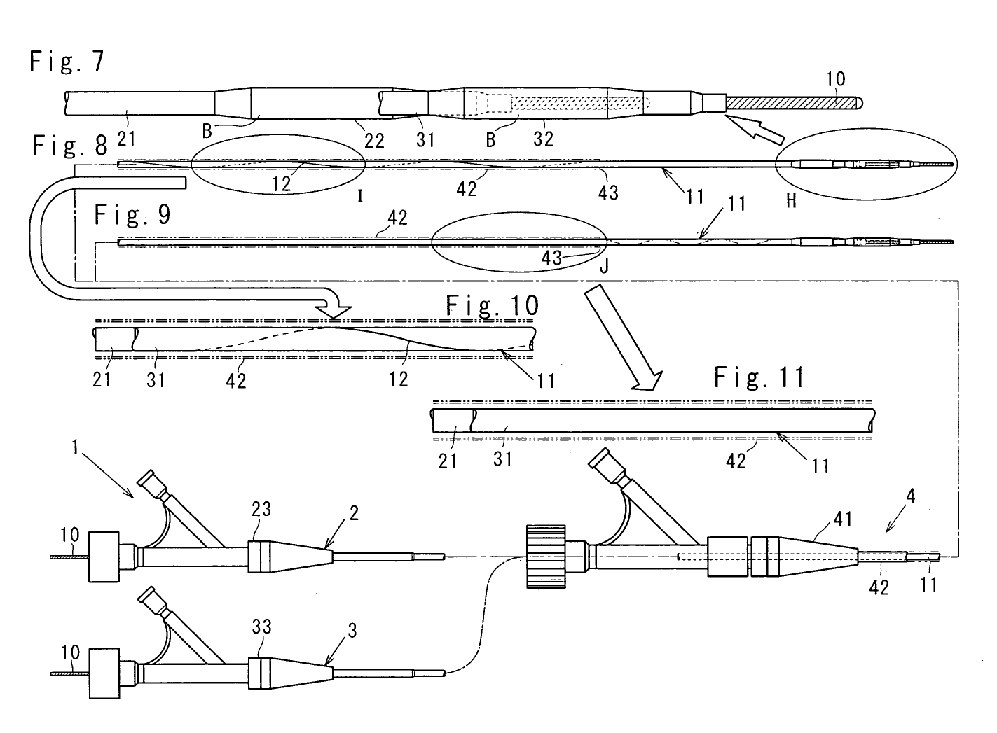

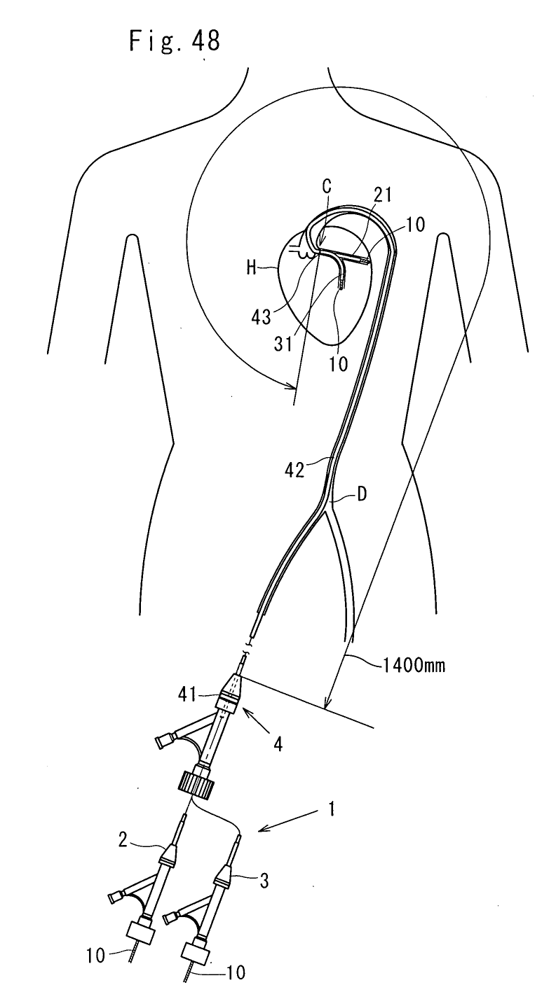

Upon using the assemble 1 of catheters as shown in FIG. 48, the assemble 1 of catheters is placed within a guiding catheter 4 (FIGS. 7-11) which is inserted into the blood vessel in advance.

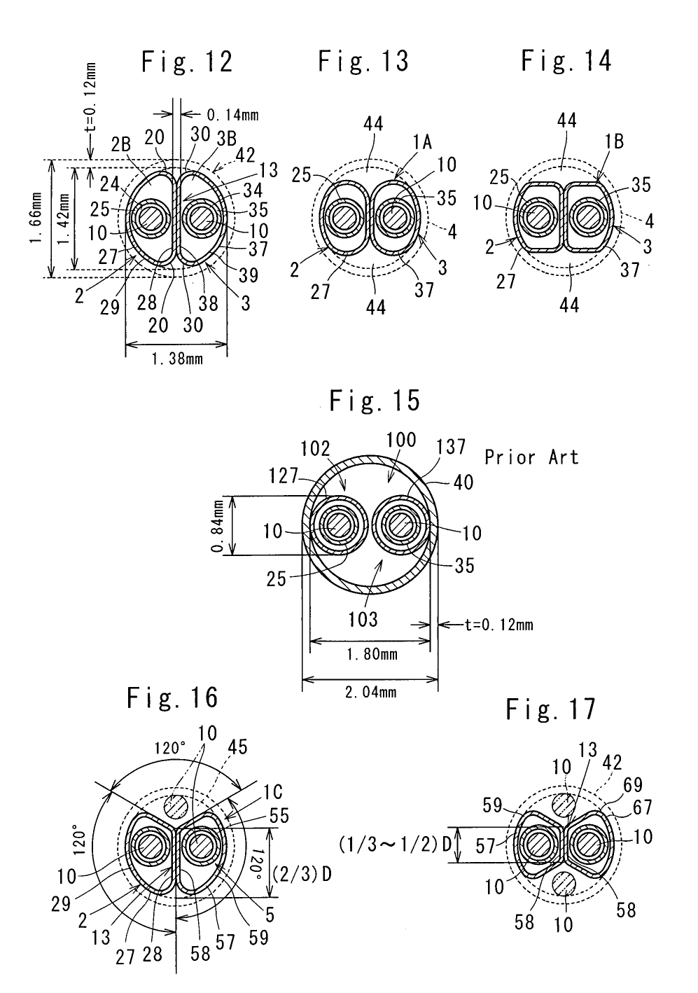

A terminal tool 41 is connected to a proximal end of the guiding catheter 4, and a flexible pipe 42 is connected to a front end of the terminal tool 41. By way of illustration, the flexible pipe 42 is circular in cross section as shown in FIG. 12. The flexible pipe 42 measures 1.66 mm (5F) in outer diameter, 1.42 mm in inner diameter and 0.12 mm in thickness.

The flexible pipe 42 measures 1400 mm in full length, so that the flexible pipe 42 reaches its distal end 43 to an entrance C of the coronary through the main artery D along a guide wire 10 which is inserted beforehand into the cardiovascular path leading to a heart H as shown in FIG. 48.

Each of the balloon catheters 2, 3 has a flexible elongate lumen tube 21 (31) and a balloon portion 22 (32), a distal end side of which is somewhat enlarged diametrically to accommodate a deflated balloon B, and further having a terminal tool 23 (33) connected to a proximal end side of the balloon catheters 2, 3 as shown in FIGS. 1 through 3.

As shown in FIGS. 5, 6, the lumen tube 21 (31) has an inner tube 25 (35) which forms an interior lumen 24 (34) to pass the guide wire 10 therethrough, and having an outer tube 27 (37) provided coaxially around the inner tube 25 (35) to form an exterior lumen 26 (36).

The exterior lumen 26 (36) is in communication with the balloon B of the balloon portion 22 (32), and develops a clearance between an outer surface of the inner tube 25 (35) and an inner surface of the outer tube 27 (37), so as to provide a balloon inflation fluid path 2B (3B) which is used to inflate and deflate the balloon B.

In the present embodiment, each of the balloon catheters 2, 3 forms the over-the-wire balloon catheter in which the guide wire 10 passes entirely through the inner tube 25 (35) and the interior lumen 24 (34).

The inner tube 25 (35) is circular in cross section, and measures 0.40 mm in inner diameter and 0.55 mm in outer diameter as best seen in FIG. 12. Into the interior lumen 24 (34), the guide wire 10 is slidably placed, an outer diameter of which measures 0.254-0.355 mm. The outer tube 27 (37) is semi-circular in cross section, and having a linear side (planar side) 28 (38) and an arcuate side (cylindrical side) 29 (39). A cornered portion 20 (30) is rounded at an inerface between the linear side 28 (38) and the arcuate side 29 (39). The outer tube 27 (37) measures 0.69 mm in outer diameter and 0.07 mm in thickness.

In the two balloon catheters 2, 3, the lumen tubes 21, 31 are juxtapositionally joined in the lengthwise direction with the linear sides 28, 38 kept in contact with each other as shown in FIGS. 2 through 4.

In this situation, an outer surface of the outer tube 27 (37) (at least the linear sides 28, 38) may be mirror-finished by means of an extrusion for a synthetic resin material with the use of a mirror-finished dice (or drawing process for a metallic tube) to have the mirror-finished surfaces joined by means of van der Waals absorption (physical absorption force). As an alternative, the outer surface of the outer tube 27 (37) may be mirror-finished by means of a polishing procedure.

As a means of joining the linear sides 28, 38, a minute projection ridge (not shown) may be provided on one of the linear sides 28, 38, and a linear cavity (not shown) may be provided on the rest of the linear sides 28, 38 to make projection ridge interfit into the cavity so as to form a rail-like interfit structure (not shown).

The outer tubes 27, 37 are slidably arranged each other along the linear sides 28, 38 in the axial direction so as to form a conjugate lumen tube 11. The conjugate lumen tube 11 is substantially circular in cross section with its outer diameter determined to be 1.38 mm, and arranged to be insertable into the guiding catheter 4, an inner diameter of which measures 1.42 mm. The conjugate lumen tube 11 is dimensionally arranged to locate near, in contact with or slidable against an inner wall of the guiding catheter 4.

The conjugate lumen tube 11, which slidably holds the outer tubes 27, 37 together in the lengthwise direction within the guiding catheter 4, renders the outer tubes 27, 37 separable at a portion {including the balloon portion 22 (32)} outside the distal end 43 of the guiding catheter 4 as shown in FIGS. 7-9.

In order to make the conjugate lumen tube 11 easily enter into the guiding catheter 4, the conjugate lumen tube 11 is twisted e.g., 1-3 times in a predetermined pitch to join the outer tubes 27, 37 in the spiral fashion as shown by a lay 12 in FIGS. 2-4. In the present embodiment, the lumen tubes 21, 31 are twisted 3 times to be helically joined to form a united single body structure.

An appropriate number of twisted turns for the conjugate lumen tube 11 is 1-3 times. When the number of twisted turns is under one time, it becomes difficult to unite the lumen tubes 21, 31 together. When the number of twisted turns exceeds three times, it becomes difficult to slidably move the lumen tubes 21, 31 each other so as to deteriorate the slidability between the lumen tubes 21, 31. It, however, is possible to ameliorate the slidability by lengthening the spiral pitch of the conjugate lumen tube 11.

The lumen tubes 21, 31 may be twisted in less than one time to be joined each other in the spiral fashion. In this instance, it is also effective in sliding one of the balloon portions 22, 32 forward or rearward while rotationally or pivotally moving one balloon portion upon implementing the kissing operation.

As shown in FIG. 9, the distal end portion of the conjugate lumen tube 11 may be twisted along the lay 12 when the lumen tubes 21, 31 are joined within the guiding catheter 4 in the lengthwise direction. The distal end portion, which extends from the distal end 43 of the guiding catheter 4, measures 80-150 mm in length. Namely, the distal end portion of the conjugate lumen tube 11, which is exposed from the guiding catheter 4 within the length of 80-150 mm at the time of implementing the therapeutical operation, may be either helically joined or linearly joined juxtapositionally.

Upon forming the helically joined structure, the lumen tubes 21, 31 may be joined after each of the lumen tubes 21, 31 helically twisted individually by means of a torsional process or a mould die. Alternatively, the conjugate lumen tube 11 may be inserted into the guiding catheter 4 after twisting conjugate lumen tube 11. Otherwise, the conjugate lumen tube 11 may be inserted into the guiding catheter 4 while progressively twisting the conjugate lumen tube 11.

Upon implementing the kissing operation, it is necessary for the balloon catheters 2, 3 to slidably move smoothly at least in the axial direction within the guiding catheter 4. For this reason, a hydrophilic polymer (e.g., polyvinyl-pyrrolidone), which exhibits a good lubricity when moistened, may be coated on an inner wall of the guiding catheter 4 and the sliding surface of the lumen tubes 21, 31 as a lubricious agent.

In FIG. 15 which shows a latitudinal cross section of a prior balloon catheter 100, to which a 6F-sized guiding catheter 40 is applied for the kissing operation, the balloon catheter 100 has two identical balloon catheters 102, 103 juxtapositionally joined together. The guiding catheter 40 measures 2.04 mm in outer diameter, 1.80 mm in inner diameter and 0.12 mm in thickness. Each of the balloon catheters 102, 103 of the same structure has corresponding outer tubes 127, 137, outer diameters of which measure 0.84 mm.

The assemble 1 of the balloon catheters-employs the guiding catheter 4, the thickness (t=0.12 mm) of which is identical to that of the prior 6F-sized balloon catheter 100. Without diametrically decreasing the inner tubes 25, 35, the assemble 1 of the balloon catheters employs the guide wire 10, an outer diameter of which is identical to that of a guide wire used when the kissing operation is implemented with the prior 6F-sized balloon catheter 100.

Despite of using the guiding catheter 4, the thickness of which is identical to that of the prior guiding catheter, and despite of using the guide wire 10, the outer diameter of which is identical to that of the prior guide wire, the 5F-sized guiding catheter 4 (1.66 mm in outer diameter) enables the user to implement the kissing operation. As described hereinafter, the 5F-sized guiding catheter 4 thus employed, ameliorates its bending rigidity, pushability and following-up capability, compared to the corresponding properties accrued from the prior guiding catheter.

The pushability means an advancing ability which the balloon catheters 2, 3 and the guide wire 10 can reach their distal ends to the diseased area in the sinuous blood vessel upon manipulatively moving the balloon catheters 2, 3 and the guide wire 10 forward within the guiding catheter 4 through the sinuous blood vessel. The follow-up capability means how the balloon catheters 2, 3 easily deforms along the diseased area in the sinuous blood vessel in response to the therapeutical operation implemented at the proximal side.

In the following description, the explanation for the balloon catheter 2 can serve as the explanation for the balloon catheter 3. The pushability corresponds to the rigidity of the lumen tubes 21, 31 upon moving the balloon catheters 2, 3 and the guide wire 10 forward through the sinuous blood vessel. The rigidity is a mathematical function of the moment of inertia. The assemble 1 of the balloon catheters 2, 3 has the moment of inertia approximately 5.2-5.6 times higher than the combination of the balloon catheters 102, 103 has in the prior 6F-sized balloon catheter 100.

The reason for the increased moment of inertia is as follows:

The conjugate lumen tube 11 has the outer tube 27 (37) semi-circular in cross section with the arcuate side 29 (39) slidably arranged against the inner wall of the guiding catheter 4, thus making it possible to diametrically enlarge the outer tube 27 (37). The outer tube 27 (37) joins the linear sides 28 (38) as lumen partitions in a face-to-face relationship with each other, so as to form a prop wall 13 (0.07×2 mm in thickness) in the diametrical direction as shown in FIG. 12. Under the presence of the prop wall 13, the conjugate lumen tube 11 has the moment of inertia approximately 5.2-5.6 times higher than the combination of the balloon catheters 102, 103 has in the prior balloon catheter 100.

Since the conjugate lumen tube 11 forms the lay 12 as a torsional line at the predetermined pitch as shown in FIG. 2, the outer tube 27 (37) renders the linear sides 28 (38) to curve in the spiral fashion around the axial direction of the outer tube 27 (37). This gradually varies the bending rigidity of the conjugate lumen tube 11 along the axial direction of the outer tube 27 (37) (gradual rigidity-transition property) without unilaterally increasing the rigidity of the conjugate lumen tube 11. It becomes possible to ameliorate the follow-up capability of the conjugate lumen tube 11 to enable the user to insert it deeply into the blood vessel by discreetly using a high-rigidity portion and a low-rigidity portion of the conjugate lumen tube 11 at the diseased area.

The assemble 1 of the balloon catheter enables the user to reduce the time period necessary to deflate the balloon during the therapeutical operation in the following reasons, thus decreasing the cessation time of the blood streams so as to avoid a symptom caused by ischemia.

After dilating the occlusive area by inflating the balloon B of the balloon portion 22 (32) of the balloon catheters 2, 3, it is necessary to deflate the balloon B in order to quickly release the cessation of the blood streams. In this instance, it also needs to retrieve the predetermined quantity of the balloon inflation liquid (e.g., physiological saline solution) for a shorter period of time.

Upon retrieving the balloon inflation liquid, it is known that the time period (deflation time) necessary to retrieve the balloon inflation liquid, reduces more as the balloon inflation fluid path 2B (3B) increases its cross sectional area more. Namely, the deflation time reduces generally with the increase of the cross sectional area of the balloon inflation fluid path 2B (3B).

Despite of using the 5F-size balloon catheters 2, 3, the assemble 1 of the balloon catheter enables the user to increase the cross sectional area of the balloon inflation fluid path 2B (3B) by approximately 1.6 times as great as that of the prior balloon catheter 100 for use in the kissing operation. This makes it possible to positively reduce the deflation time period necessary to retrieve the balloon inflation liquid.

FIGS. 13 and 14 in turn show latitudinal cross sectional views with respect to assembles 1A, 1B of balloon catheter of second and third embodiments of the invention.

In the second and third embodiments of the invention, the outer tubes 27, 37 of the second embodiment increase its curvature at the cornered portions of the latitudinal cross section of the assemble 1A of balloon catheters. The outer tubes 27, 37 of the third embodiment arrange an upper side in parallel with a lower sides of the latitudinal cross section of the assemble 1B of balloon catheters.

The cross section of the outer tubes 27, 37, according to the second embodiment, makes an outer arcuate side contact with an inner wall of the guiding catheter 4 to generally form an oval or elliptical configuration. The cross section of the outer tubes 27, 37, according to the third embodiment, makes an outer arcuate side contact with an inner wall of the guiding catheter 4 to shape a rectangle-deformed configuration.

Upon implementing the kissing operation, after dilating the occlusive area, a contrast medium may be applied to the guiding catheter 4 in order to confirm an dilated condition of the occlusive area through a fluorography.

Within the guiding catheter 4, an exterior space surrounding the outer tubes 27, 37 serves as a delivery path 44 for the contrast medium. It is a matter for a design adjustment to appropriately determine a latitudinal cross sectional area occupied by the delivery path 44 while taking a latitudinal cross sectional area of the balloon inflation fluid path 2B (3B) into consideration.

The following procedures are taken upon implementing the kissing operation as a method of inserting the balloon catheter.

To outer surfaces of the two guide wires 10 admitted into the blood vessel, the balloon catheters 2, 3 insert their lumen tubes 21, 31 through its proximal end portion extracorporeally exposed.

In this instance, the lumen tubes 21, 31 may be joined beforehand, otherwise the separately placed lumen tubes 21, 31 may be joined at an open entrance of the terminal tool 41 of the guiding catheter 4. At the time of joining the lumen tubes 21, 31, the lumen tubes 21, 31 may be juxtaposed side by side in the axial direction as shown in FIG. 2, otherwise the lumen tubes 21, 31 joined together may be twisted around the axial direction in the spiral fashion at the predetermined pitch as shown in FIG. 2.

In this situation, the balloon portions 22, 32 are placed offset in a front-and-rear relationship with each other, so as to prevent the balloon portions 22, 32 from being overlapped side by side at a common position.

Since each of the balloon portions 22, 32 measures 0.83 mm in outer diameter, a unit of the two balloon portions 22, 32 measures 1.66 mm in outer diameter when the two balloon portions 22, 32 are arranged to be overlapped side by side, thus making it impossible to insert it into the 5F-sized guiding catheter 4, an inner diameter of which measures 1.38 mm.

The balloon catheter 2 places the balloon portion 22 at the proximal side remote from the balloon portion 32 to make the balloon portion 22 engage against a distal end of the outer tube 37 so as to deform the balloon portion 22 in less than 1.38 mm in outer diameter.

This makes it possible to use the balloon B for the 5F-sized guiding catheter incorporated into the assemble of balloon catheters, even when the balloon B has the same outer diameter as that of the balloon of the prior balloon catheter used for the 6F-sized guiding catheter 40.

Into the guiding catheter 4 which is beforehand admitted into the blood vessel, the conjugate lumen tube 11 is inserted to lead the balloon portions 22, 23 to the diseased area as a target destination.

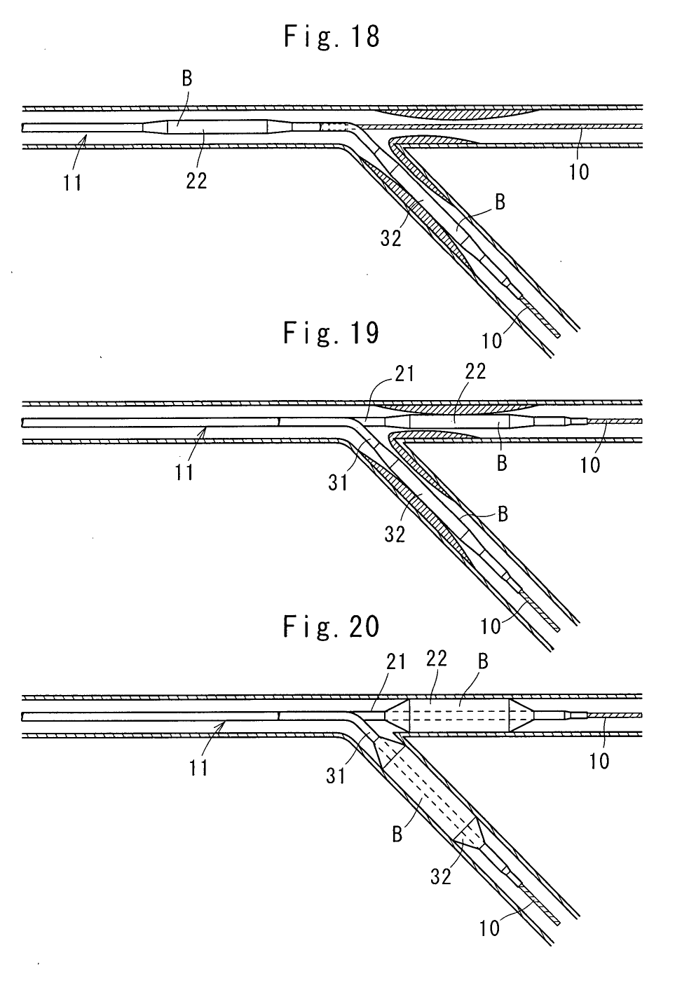

Then, one of the balloon catheters 2, 3 is operated to introduce the balloon 32 into the diseased area of one (e.g., lateral branch) of the bifurcated blood vessel as shown in FIG. 18.

Thereafter, the other balloon portion 22 is operated to be introduced into the diseased area of the main trunk of the blood vessel as shown in FIG. 19. Especially when the two balloon catheters 2, 3 are joined in the spiral fashion, the manual operation enables the user to advance the balloon catheters 2, 3 forward with the rotational movement accompanied, thus making it possible to arbitrarily change the route in the blood vessel, so as to easily enter the conjugate lumen tube 11 into the occlusive area of the blood vessel.

By way of the terminal tools 23, 33, the physiological saline solution is supplied to the balloon portions 22, 23 so as to inflate the two balloons B at the same time as shown in FIG. 20.

After an elapse of the needed time period, a manual operation deflates the two balloons B to be retrieved. If the fluorography is needed to confirm the dilated condition of the occlusive area, the procedure is taken to infuse the contrast medium into the remedied portion of the bifurcated blood vessel through the delivery path 44 before retrieving the two balloons B.

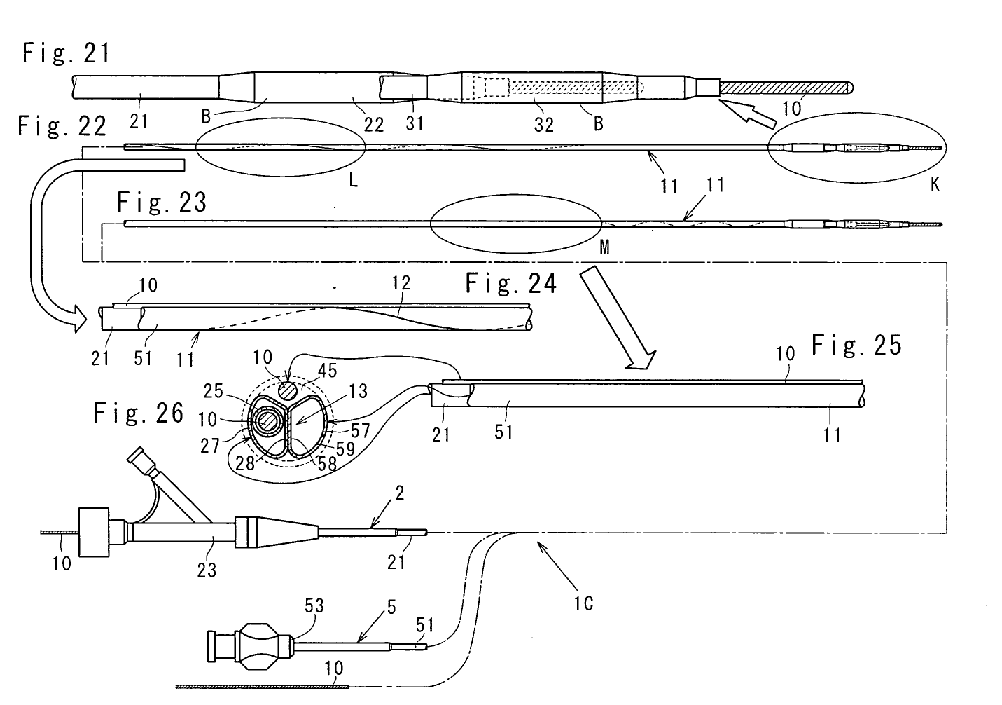

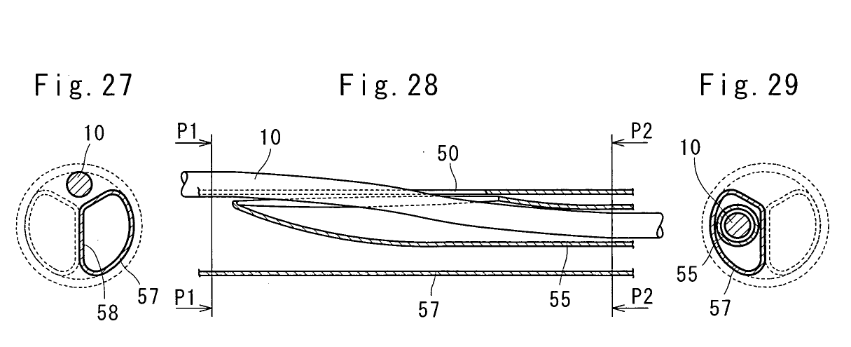

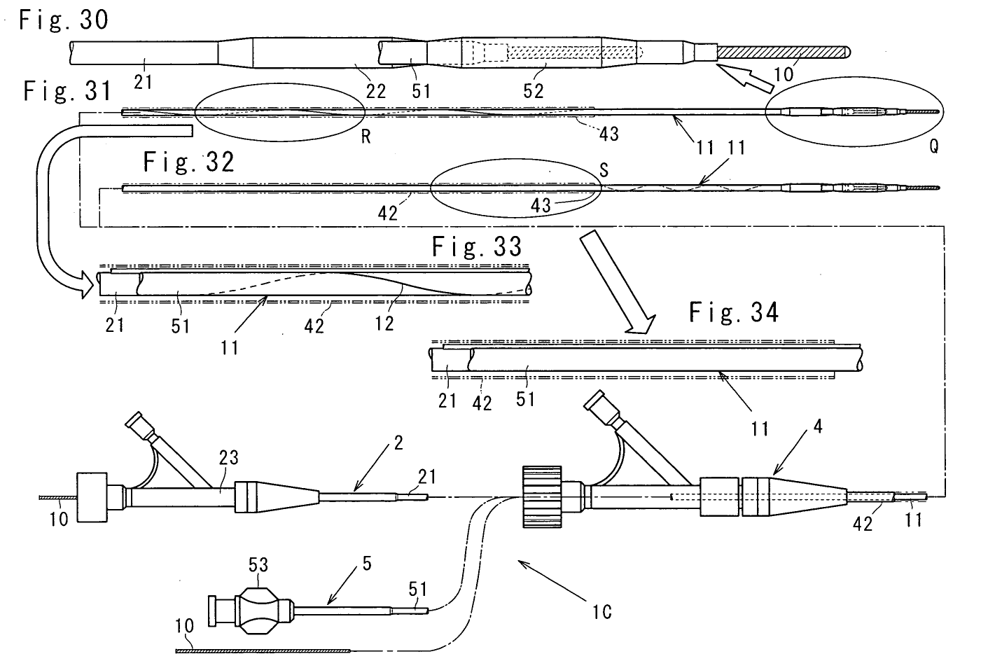

FIGS. 21 through 26 show a fourth embodiment of the invention in which an assemble 1C of catheters is provided. FIGS. 27 and 29 are left and right enlarged latitudinal cross sectional views of main portions of the assemble 1C of catheters. FIG. 28 is an enlarged longitudinal cross sectional view of the main portion of the assemble 1C of catheters. FIGS. 30 through 34 show schematic view of the assemble 1C of catheters which is inserted into the guiding catheter 4.

In the fourth embodiment of the invention, the over-the-wire balloon catheter 2 and the rapid-exchange (RX) balloon catheter 5 are joined together, basic structures of which are known for those versed in the art.

The balloon catheter 5 has a flexible elongate lumen tube 51 and a somewhat diametrically enlarged balloon portion 52, a distal end portion of which accommodates the deflated balloon B. A proximal end portion of the balloon catheter 5 has a terminal tool 53.

As shown in FIGS. 16, 26, the outer tube 27 (57) (fan-like in cross section) has the linear portion 28 (58), a length of which is approximately ⅔ of the outer diameter (D) of the outer tube 27 (57) with the arcuate portion (semi-cylindrical portion) 29 (59) having approximately 120 degrees as the angle at the circumference.

The conjugate lumen tube 11, which is defined by linearly juxtapositionally or helically joining the outer tubes 27, 57 along the lay 12, forms its latitudinal cross section into a deformed fan-like configuration with an arc portion being approximately 240 degrees as the angle at the circumference. A space surrounded by the rest arc portion, which occupies approximately 120 degrees as the angle at the circumference, forms a contrast medium supply path 45 or a passageway for the guide wire 10 of the rapid-exchange balloon catheter 5.

At the proximal side, the balloon catheter 5 juxtaposes the guide wire 10 with the outer tube 57 outside as shown in FIG. 28, and setting the guide wire 10 into an inner tube 55 coaxially placed within the outer tube 57 through an opening 50 provided on the outer tube 57 at the distal side. That is to say, the guide wire 10 is arranged to pass through the inner tube 55 at the distal side to make the distal end jut out of a distal end of the inner tube 55. Namely, the elongate lumen tube 51 has the inner tube 55, the distal end of which serves as an exit opening for the guide wire 10, and having the outer tube 57 coaxially provided around the inner tube 55, the distal end side of the outer tube 55 has an outer insertion opening for the guide wire 10 which is to be in communication with an inner insertion opening for the guide wire 10 and the balloon B of the balloon portion 22 (32).

Since the conjugate lumen tube 11 has the prop wall 13 as lumen partitions at the central cross sectional position in the diametrical direction as described in the first embodiment of the invention, the prop wall 13 gains a moment of inertia for the conjugate lumen tube 11 to secure a high rigidity.

Since the conjugate lumen tube 11 insures substantially the same latitudinal cross sectional area for the balloon inflation fluid path of the prior kissing balloon catheter 100 (6F), the conjugate lumen tube 11 obtains substantially the same deflation time as that of the prior kissing balloon catheter 100.

For this reason, the assemble 1C of balloon catheters enables the user to hold the same functions as those of the prior kissing balloon catheter 100 when the assemble 1C of balloon catheters is downsized to 5F size in scale.

Upon implementing the kissing operation, it is possible for the balloon catheters 2, 5 to attain the same maneuverability as the balloon catheters 2, 3 achieves in the first embodiment of the invention.

Since a delivery path 45 provided for the contrast medium, it is possible to confirm the dilated state through the fluorography after dilating the occlusive area.

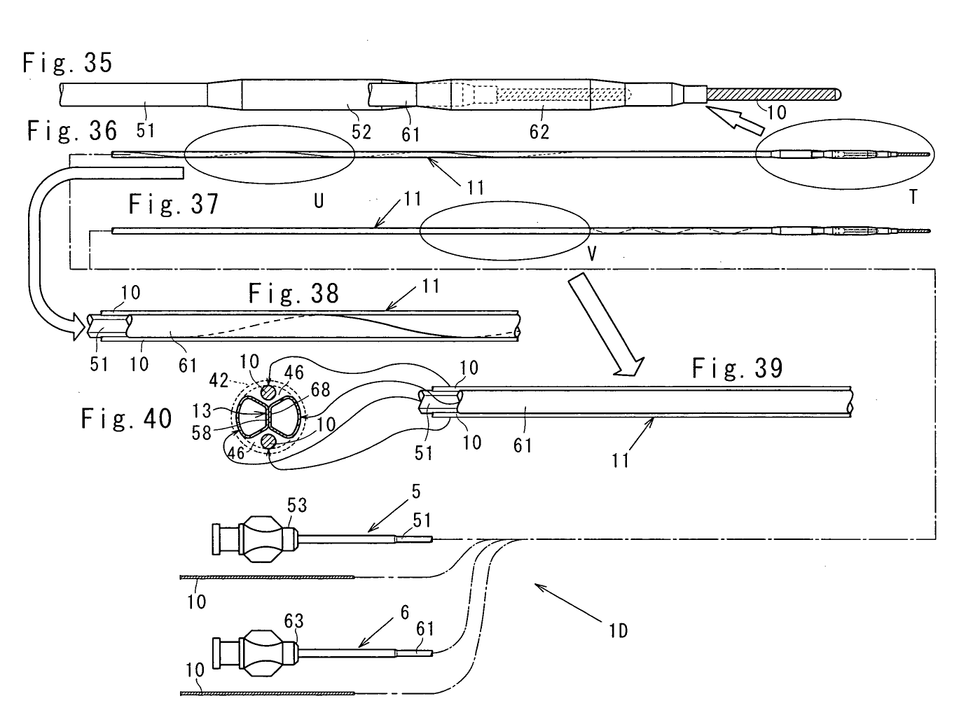

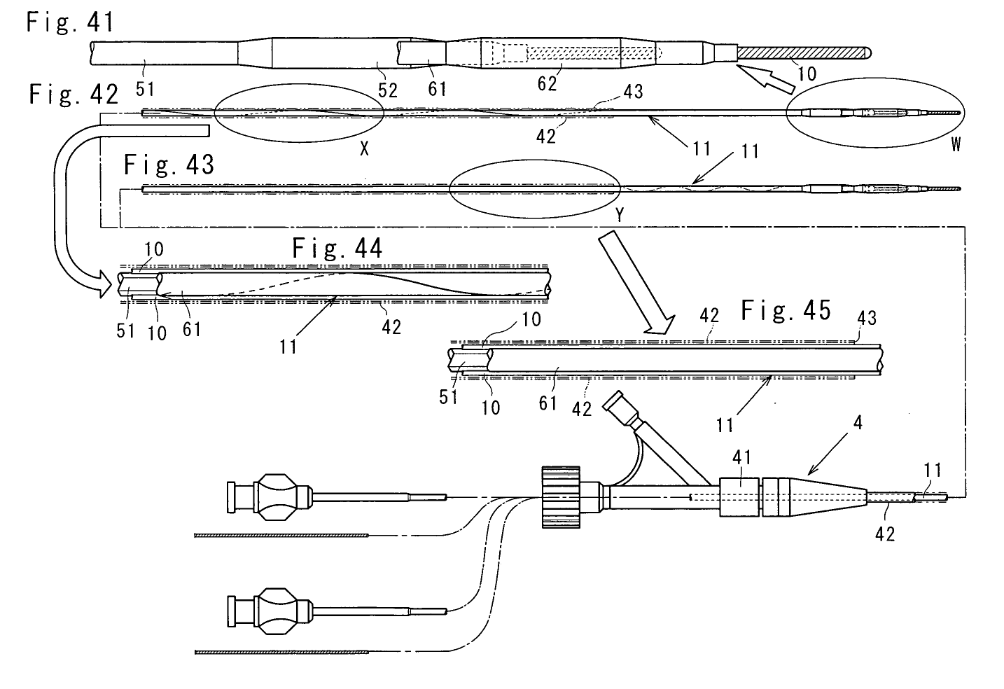

FIGS. 35 through 45 show a fifth embodiment of the invention in which an assemble 1D of catheters is provided. The assemble 1D of catheters joins two rapid-exchange balloon (RX) catheters 5, 6 together. The balloon catheter 6 has a flexible elongate lumen tube 61 and a somewhat diametrically enlarged balloon portion 62, a distal end of which accommodates the deflated balloon B. A terminal tool 63 is secured to the proximal side of the balloon catheter 6.

As shown in FIGS. 17, 40, the balloon catheter 5 (6) defines the respective cross section of the outer tube 57 (67) substantially as a trapezoidal configuration (or fan-like configuration) with an outer arcuate portion (semi-cylindrical portion) 59 (69) arcuately formed along the inner wall of the guiding catheter 4.

In the balloon catheter 5 (6), the lumen tube 51 (61) has an inner planar side (shorter side) 58 (68), a length of which measures approximately ⅓-½ of an outer diameter of the lumen tube 51 (61). The lumen tube 51, 61 are juxtapositionally joined in the lengthwise direction or twisted with the lateral planar sides 58, 68 abutted against each other. The conjugate lumen tube 11 has a space 46 at an upper portion and a lower portion. The space 46, which serves as the passageway for the guide wire 10, has a latitudinal cross section which is shaped into a fan-like configuration with the angle at the circumference defined approximately 120 degrees as shown in FIG. 40.

Since the assemble 1D of balloon catheters has the prop wall 13 at the central cross sectional position of the conjugate lumen tube 11 in the diametrical direction as described in the assemble 1C of balloon catheters, the prop wall 13 increases the moment of inertia for the conjugate lumen tube 11 to secure a high rigidity.

Since the conjugate lumen tube 11 insures substantially the same latitudinal cross sectional area for the balloon inflation fluid path of the prior kissing balloon catheter 100 (6F), the conjugate lumen tube 11 obtains substantially the same deflation time as that of the prior kissing balloon catheter 100.

For this reason, the assemble 1C of balloon catheters enables the user to hold the same functions as those of the prior kissing balloon catheter 100 when the assemble 1C of balloon catheters is downsized to 5F size in scale. Upon implementing the kissing operation, it is possible for the balloon catheters 2, 5 to attain the same maneuverability as the balloon catheters 2, 3 achieve in the first embodiment of the invention.

Since the space 46 serves as the delivery path for the contrast medium, it is possible to smoothly confirm the dilated condition by way of the fluorography.

The following are advantages obtained by joining the lumen tubes 21, 31 (outer tubes 27, 37) of the two balloon catheters 2, 3 in the spiral fashion.

The conjugate lumen tube 11 has an extension portion which is to be inserted into the guiding catheter 4, and at least the extension portion is twisted 1-3 times as shown at the lay 12 in FIG. 44. This enables the user to smoothly insert the conjugate lumen tube 11 into the guiding catheter 4.

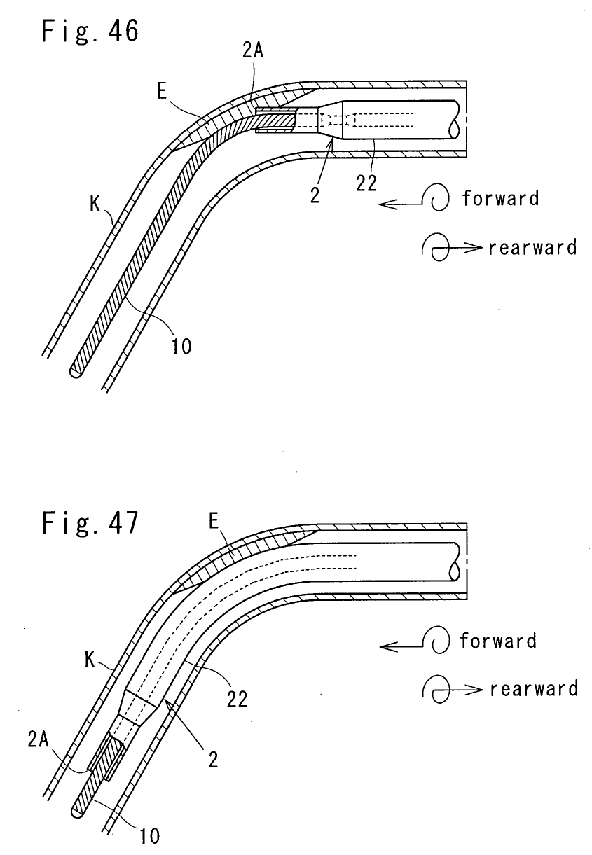

Upon inserting the conjugate lumen tube 11 into the diseased area, it is possible to advance one of the balloon catheters 2, 3 forward while rotating the same balloon catheter around its lengthwise direction. Even when the balloon catheter 2 makes its distal end portion 2A hitch at the diseased tissue portion E as shown in FIG. 46, it is possible to dislodge the distal end portion 2A from the hitch of the diseased tissue portion E by rotationally moving the distal end portion 2A forward or rearward as shown in FIG. 47.

By developing the technological idea of the present invention, it is possible to diametrically thin the assemble 1 (1A, 1B, 1C, 1D) of the catheters. By way of example, it is possible to thin the 5F-sized guiding catheter 4 into a 4F-sized guiding catheter, an outer diameter of which generally measures 1.33 mm.

In more specific, with the use of the guide wire 10 having 0.203-0.254 mm (0.008-0.010 inches) in outer diameter, inner and outer diameters of the inner tube ranges from 0.320 mm to 0.425 mm, and an outer diameter of the outer tube measures 0.525 mm when semi-circular in cross section, so as to render the conjugate lumen tube 11 to be 1.05 mm in outer diameter.

In this situation, the 4F-sized guiding catheter 4 substantially measures 1.33 mm in outer diameter, and 1.09 mm in inner diameter even with the 4F-sized guiding catheter 4 has the same thickness (t 0.12 mm) as the 6F-sized guiding catheter 40 has in the assemble of balloon catheters.

This makes it possible to insert the conjugate lumen tube 11 (1.05 mm in outer diameter) into the 4F-sized guiding catheter. In this way, the technological idea of the present invention leads to the assemble of thinned balloon catheters used for the kissing operation.

The prior balloon catheter 100 for the kissing operation has a metallic core inserted along an annular space between the inner tube 25 and an outer tube 127. The metallic core is diametrically reduced progressively as approaching forward, and inserted into the annular space by approximately 250 mm from the proximal side to the distal side so as to enhance a pushability of the balloon catheter 100.

To the proximal end side of the outer tube 127 in the balloon catheter 100 used for the kissing operation, the synthetic resin (polyimide) have been applied with inner and outer diameters as 0.75 mm and 0.84 mm. In recent years, instead of the synthetic resin tube, employed is a stainless steel hypotube which has inner and outer diameter as 0.43 mm×0.67 mm so as to enhance the pushability of the balloon catheter.

By employing the metallic hypotube to the proximal end side of the outer tube 27 (37) instead of the synthetic resin tube, and forming the latitudinal cross section of the outer tube 27 (37) substantially into the semi-circular configuration, it is also possible to provide the assemble 1 (1A, 1B, 1C, 1D) of further thinned balloon catheters.

The following are descriptions to ameliorate the slidability and prevent the thrombi from depositing on the outer tubes 27 (37) in connection with the juxtapositionally or helically joined lumen tubes 21, 31 (outer tubes 27, 37).

(a) Ameliorated Slidability on Lumen Tubes

In the assemble 1 of balloon catheters according to the present invention, there are provided the two outer tubes 27, 37, each of which is substantially similar in latitudinal cross section. The kissing operation moves the two outer tubes 27, 37 forward or rearward in the lengthwise direction by sliding the juxtapositionally joined side surfaces thereof. Otherwise, the kissing operation rotationally moves the two outer tubes 27, 37 forward or rearward in the spiral fashion by sliding helically joined side surfaces thereof.

This makes the two outer tubes 27, 37 move each other by sliding the same kind of material members, thus inducing agglutination and stick-slip phenomenon at the joined side surfaces to likely hinder a smooth kissing operation.

As the raw material of the outer tubes 27, 37, used are polypropylene (PP), polyamide (PA), fluoroplastic (PFA, etc), polytetrafluoroethylene (4F:PTFE), polycarbonate (PC), polyethylene and polyimide.

Among those raw materials, there are members which exhibit an exceptionally high slidability. Upon conducting a sliding experimentation, gradient angles are measured when each of the raw material test pieces begins to fall along an oblique slide surface.

The gradient angles are converted into static coefficients of friction as shown in Table. The raw material test pieces generally tends to exhibit higher static coefficients of friction between the identical kind of material members while exhibiting lower static coefficients of friction between the different kind of material members.

TABLE

Mobile side

| Fixed side | PC | PP | PA | 4F |
|---|---|---|---|---|
| PC | 0.524 | 0.363 | 0.227 | 0.200 |
| PP | 0.360 | 0.393 | 0.383 | 0.224 |
| PA | 0.484 | 0.445 | 0.731 | 0.380 |
| 4F | 0.173 | 0.252 | 0.283 | 0.134 |

Among the fluoroplastic materials, polytetrafluoroethylene (4F:PTFE) is contrary to the above tendency because it represents the least static coefficient of friction between the identical kind of material members. This teaches that it is preferable to use polytetrafluoroethylene (4F:PTFE) for the joined side surfaces of the outer tube 27 (37).

In more tangible terms, it is preferable to apply a polytetrafluoroethylene coating to the sliding or engagement portion of the joined side surfaces of the outer tube 27 (37) when the metallic tube {e.g., hypotube (stainless steel tube)} is used to the outer tube 27 (37).

In this sense, it becomes possible to insure a high slidability and smooth maneuverability for the balloon catheters by applying the same kind of sliding material to both the outer tubes 27, 37.

(b) Thrombi Prevented from Depositing on Outer Tubes

It is preferable to coat antithrombotic synthetics on the outer surface of the outer tubes of the balloon catheters in order to exclude the balloon catheters from losing a good maneuverability due to the thrombi appeared within the guiding catheter.

As the antithrombotic synthetics, preferably used are polyhydroxyethyl-methacrylate, styrene-hydroxyethylmethacrylate copolymer and the equivalents.

(c) Materials for Balloon Catheter

As materials for use in the inner tube which is arranged to be slidably against the guide wire, preferable are polyamide, polyethylene, fluoroplastic, polypropylene and the equivalents.

(d) As materials for use in the outer tube which serves as the balloon inflation fluid path for the balloon catheters, polyamide, polyethylene, fluoroplastic, polyimide and the equivalents. As the metallic material, used are stainless steel, nickel, superelastic alloy (titanium-based alloy, etc.). Especially in order to ameliorate the pushability, it is preferable to use the metallic material such as, for example, stainless steel at the proximal side of the outer tube.
(e) As materials for use in the balloon portion, used are polyamide, polyethylene, polyesterelastomer and the equivalents.
(f) As materials for use in the end side (forked portion) and the proximal side of the balloon catheters, used are polycarbonate, polysulfone, polyacrylate and the equivalents.

Connecting Structure of Outer Tube

A concave groove may be provided at an open end of the outer tube of high rigidity so as to strengthen the connection between the connected tubes. The rapid-exchange balloon catheter uses the hypotube as the inner tube of high rigidity extending 250-300 mm from a distal end (in the proximity of an entrance of the guide wire) to the proximal side of the guide wire.

In this instance, the balloon catheter arranges the front portion of the outer tube along the distal end side of the balloon portion, and forms the front portion of the outer tube by the plastic tube connected to the hypotube.

At the connected tube portion in which the outer tube is circular, oval or elliptical in cross section, a metallic tube end of high rigidity may be roughly machined to be tapered, and connected to a plastic tube of middle rigidity by means of a thermal bonding procedure, in order to alleviate the property in which the rigidity changes abruptly at a certain place of the outer tube.

In this situation, a front plastic (flexible) tube may be connected to a tube of middle rigidity.

While several illustrative embodiments of the invention have been shown and described, numerous variations and alternate embodiments will occur to those skilled in the art. Such variations and alternate embodiments are contemplated, and can be made without departing from the spirit and scope of the invention as defined in the appended claims.

1-16. (canceled)

17. In a balloon catheter which includes an elongate lumen tube, a balloon portion provided at a distal end of said elongate lumen tube and a terminal tool provided at a proximal end of said balloon catheter, so as to be inserted into a guiding catheter which is to be disposed in a blood vessel when in use;
said balloon catheter forming over-the-wire balloon catheters in which said elongate lumen tube has an inner tube into which a guide wire is inserted, and having an outer tube provided around said inner tube to be in communication with a balloon of said balloon portion; and
a latitudinal cross section of said outer tube being such as to partly form an arcuate side generally having a curvature near to a curvature of an inner wall of said guiding catheter, and partly forming a linear side at a central portion opposite to said arcuate side.

18. In a balloon catheter which includes an elongate lumen tube, a balloon portion provided at a distal end of said elongate lumen tube and a terminal tool provided at a proximal end of said balloon catheter, so as to be inserted into a guiding catheter which is to be disposed in a blood vessel when in use;
said balloon catheters forming rapid-exchange balloon catheters in which a distal end side of said elongate lumen tube has an inner insertion opening for a guide wire, an inner space of said elongate lumen tube forms an interior lumen to admit said guide wire therein;
said elongate lumen tube having an inner tube, a distal end of which serves as an exit opening for said guide wire, and having an outer tube coaxially provided around said inner tube, a distal end side of said outer tube having an outer insertion opening for said guide wire which is to be in communication with said inner insertion opening for said guide wire and a balloon of said balloon portion; and
a latitudinal cross section of said outer tube being such as to partly form an arcuate side generally having a curvature near to a curvature of an inner wall of said guiding catheter, and partly forming a linear side at a central portion opposite to said arcuate side.

19. An assembly of a balloon catheter having two balloons, said balloon catheter including an elongate lumen tube, a balloon portion provided at a distal end of said elongate lumen tube and a terminal tool provided at a proximal end of said balloon catheter, so as to be inserted into a guiding catheter which is to be disposed in a blood vessel when in use;
said balloon catheter forming over-the-wire balloon catheters in said elongate lumen tube has an inner tube into which a guide wire is inserted, and having an outer tube provided around said inner tube to be in communication with a balloon of said balloon portion;
a latitudinal cross section of said outer tube being such as to partly form an arcuate side generally having a curvature near to a curvature of an inner wall of said guiding catheter, and partly forming a linear side at a central portion opposite to said arcuate side; and
said two elongate lumen tubes being slidably joined mutually to form a conjugate lumen tube, said outer tubes of said two elongate lumen tubes being generally symmetrical or identical each other, the said two elongate lumen tubes having said inner tubes, into which said guide wire is inserted, and latitudinal cross sections of said two elongate lumen tubes are such that said outer tubes make said arcuate side slidable against or located near said inner wall of said guiding catheter.

20. An assembly of a balloon catheter having two balloons, said balloon catheter including an elongate lumen tube, a balloon portion provided at a distal end of said elongate lumen tube and a terminal tool provided at a proximal end of said balloon catheter, so as to be inserted into a guiding catheter which is to be disposed in a blood vessel when in use;
said balloon catheters forming rapid-exchange balloon catheters in which a distal end side of said elongate lumen tube has an inner insertion opening for a guide wire, an inner space of said elongate lumen tube forms an interior lumen to admit said guide wire therein;
said elongate lumen tube having an inner tube, a distal end of which serves as an exit opening for said guide wire, and having an outer tube coaxially provided around said inner tube, a distal end side of said outer tube having an outer insertion opening for said guide wire which is to be in communication with said inner insertion opening for said guide wire and a balloon of said balloon portion;
a latitudinal cross section of said outer tube being such as to partly form an arcuate side generally having a curvature near to a curvature of an inner wall of said guiding catheter, and partly forming a linear side at a central portion opposite to said arcuate side; and
said two elongate lumen tubes being slidably joined mutually to form a conjugate lumen tube, said outer tubes of said two elongate lumen tubes being generally symmetrical or identical each other, the said two elongate lumen tubes having said inner tubes, into which said guide wire is inserted, and latitudinal cross sections of said two elongate lumen tubes are such that said outer tubes make said arcuate side slidable against or located near said inner wall of said guiding catheter.

21. An assembly of the balloon catheters according to claim 19 or 20, wherein said conjugate lumen tube located at least within said guiding catheter is formed by linearly juxtapositionally joining or helically joining said two elongate lumen tubes.

22. An assembly of the balloon catheters according to claim 19 or 20, wherein both said balloon catheters are over-the-wire balloon catheters.

23. An assembly of the balloon catheters according to claim 22, wherein said two balloon catheters are said over-the-wire balloon catheters, latitudinal cross sections of said outer tubes being formed into a semi-circular, oval or elliptical configuration.

24. An assembly of the balloon catheter according to claim 19 or 20, wherein one of said two balloon catheters is an over-the-wire balloon catheter, and the other of said two balloon catheters is a rapid-exchange balloon catheter.

25. An assembly of the balloon catheters according to claim 19 or 20, wherein one of said two balloon catheters is an over-the-wire balloon catheter with the latitudinal cross section of said outer tube formed into a fan-shaped configuration, and the other of said two balloon catheters is a rapid-exchange balloon catheter with the latitudinal cross section of said outer tube formed into a fan-shaped configuration.

26. An assembly of the balloon catheters according to claim 19 or 20, wherein both said balloon catheters are rapid-exchange balloon catheters.

27. An assemble of the balloon catheter according to claim 26, wherein said two balloon catheters are said rapid-exchange balloon catheters, latitudinal cross sections of said outer tubes being generally formed into a trapezoidal configuration with one lateral side shaped arcuately.

28. An assembly of the balloon catheter according to claim 19 or 20, wherein balloon portions of two balloon catheters are juxtaposed in a front-and-rear relationship with each other, so as to prevent said balloon portions from being overlapped side by side at a common position.

29. An assembly of the balloon catheters according to claim 19 or 20, wherein an outer diameter of said guide wire is 0.254-0.355 mm (0.010-0.014 inches), and an outer diameter of said guiding catheter is approximately 1.66 mm (5F).

30. An assembly of the balloon catheters according to claim 18 wherein an outer diameter of said guide wire is 0.203-0.254 mm (0.008-0.010 inches), and an outer diameter of said guiding catheter is approximately 1.33 mm (4F).

31. A method of inserting an assemble of balloon catheters into a guiding catheter comprising steps of:
inserting a guiding catheter into a blood vessel along two guide wires inserted into said blood vessel, proximal end sides of said two guide wires are extracorporeally exposed;
inserting the two balloon catheters according to claim 18 to outer surfaces of said two guide wires from the corresponding distal ends of said balloon portions through said two guide wires exposed at a proximal end side of said guiding catheter;
linearly juxtapositionally joining or helically joining said two elongate lumen tubes of said balloon portions to form a conjugate lumen tube; and
inserting said conjugate lumen tube into said guiding catheter after forming said conjugate lumen tube.
